(19) United States
(12) Patent Application Publication
(10) Pub. No.: US 2006/0229678 A1
(43) Pub. Date: Oct. 12, 2006

(54) Computer mouse with transcutaneous electro nerve stimulation capabilities

(75) Inventor: Kuo-Vicent Lee, Taipei (TW)

Correspondence Address: BACON & THOMAS, PLLC, 625 SLATERS LANE, FOURTH FLOOR, ALEXANDRIA, VA, 22314, US

(73) Assignee: BENEXT INNO-PRODUCT DEVELOPMENT LTD., Taipei (TW)

(21) Appl. No.: 11/183,830

(22) Filed: Jul. 19, 2005

(30) Foreign Application Priority Data
Apr. 12, 2005 (TW) 094111433

(51) Int. Cl. A61N 1/18

(52) U.S. Cl. 607/2

(57) ABSTRACT

A computer mouse with transcutaneous electro nerve stimulation capabilities includes two major devices: a computer mouse device, and a transcutaneous electro nerve stimulator (T.E.N.S.), both of which use a common casing. A first conductive region and a second conductive region are disposed on the surface of the common casing, so that the user of the computer mouse can benefit from the T.E.N.S.

### BACKGROUND OF THE INVENTION

1. Field of the Invention

The present invention relates to a computer mouse with transcutaneous electro nerve stimulation capabilities.

2. Description of the Related Art

Computer users may develop tendonitis in the wrist after extensive use of a computer; therefore, it is desirable to prevent or reduce wrist tendonitis problems. U.S. Pat. No. 6,323,841 utilizes a vibrating massage device on a computer mouse that enables users to use the massage device to massage their hands. However, a user is unable to operate the computer mouse and the massage device at the same time, because it is difficult to operate the computer mouse when the vibration device is turned on.

Therefore, it is desirable to provide a computer mouse with transcutaneous electro nerve stimulation capabilities to mitigate and/or obviate the aforementioned problems.

### SUMMARY OF THE INVENTION

A main objective of the present invention is to provide a computer mouse with transcutaneous electro nerve stimulation capabilities for a user.

Another objective of the present invention is to provide a computer mouse with transcutaneous electro nerve stimulation capabilities to reduce wrist tendonitis problems.

In order to achieve the above-mentioned objectives, a computer mouse with transcutaneous electro nerve stimulation capabilities of the present invention has the shape of a typical computer mouse. The computer mouse with transcutaneous electro nerve stimulation capabilities includes two major devices: a computer mouse device, and a transcutaneous electro nerve stimulator, both of which use a common casing. A first conductive region and a second conductive region are disposed on the surface of the common casing.

A first output electrode and a second output electrode of the transcutaneous electro nerve stimulator are separately connected to the first conductive region and the second conductive region, so when the user is holding the device, the palm of the user can touch first conductive region and the second conductive region. According to study, the lao-gong point in human palm is a very important point (according to traditional Chinese medical science, by stimulating the lao-gong point, the wrist tendonitis problems and other shoulder and neck problems can all be reduced), therefore, the first conductive region should be placed at a position that the lao-gong point in the palm can touch.

Other objects, advantages, and novel features of the invention will become more apparent from the following detailed description when taken in conjunction with the accompanying drawings.

### BRIEF DESCRIPTION OF THE DRAWINGS

FIG. 1 is a perspective drawing of a first embodiment of a computer mouse with transcutaneous electro nerve stimulation capabilities according to the present invention.

FIG. 2 is a schematic cross-sectional drawing of the first embodiment of the combined computer mouse and transcutaneous electro nerve stimulator.

FIG. 3 shows a position of lao-gong point.

FIG. 4 is a perspective drawing of a second embodiment of a computer mouse with transcutaneous electro nerve stimulation capabilities according to the present invention.

FIG. 5 is a perspective drawing of a third embodiment of a computer mouse with transcutaneous electro nerve stimulation capabilities according to the present invention.

### DETAILED DESCRIPTION OF THE PREFERRED EMBODIMENT

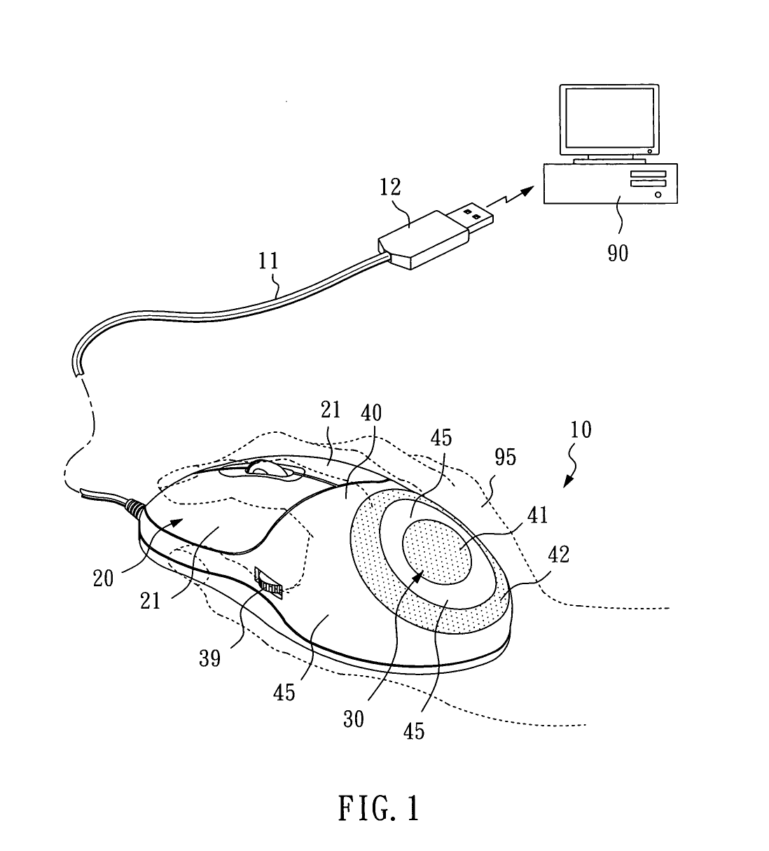

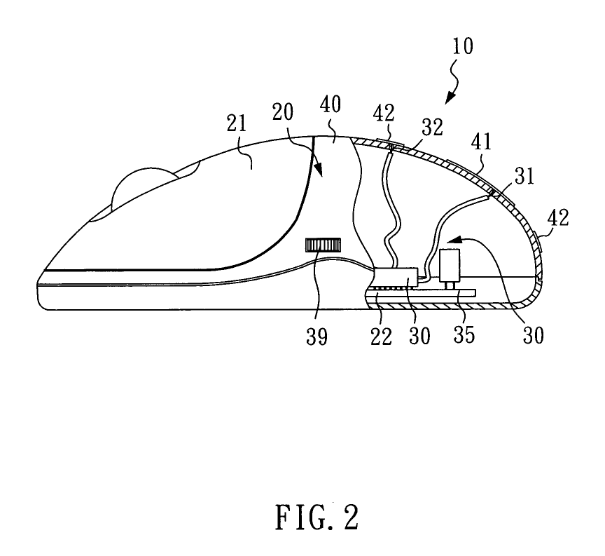

Please refer to both FIG. 1 and FIG. 2. In a first embodiment, a computer mouse with transcutaneous electro nerve stimulation capabilities 10 has a shape typical of computer mice. The computer mouse with transcutaneous electro nerve stimulation capabilities 10 comprises two major devices: a computer mouse device 20 and a transcutaneous electro nerve stimulator 30.

The computer mouse device 20 can be connected to a computer 90 via a transmission cable 11; in the first embodiment, a USB (Universal Serial Bus) connector 12 is used for the connection, and because of the characteristics inherent in the USB standard the computer 90 can supply power to the transcutaneous electro nerve stimulator 30. The computer mouse device 20 comprises a mechanical device (such as mouse buttons 21) and an electronic device 22 (such as the typical mouse circuit, which may include a USB control circuit). Since the computer mouse is a well-known technology, it requires no further description; however, it should be noted that the computer mouse device 20 may be a roller-type computer mouse or an optical computer mouse.

The transcutaneous nerve electro stimulator 30 (T.E.N.S.) outputs a low frequency pulse to stimulate nerves (or a point) in the human body. A circuit 35 of the transcutaneous electro nerve stimulator 30 utilizes a wave form generator and two output electrodes; however, various designs of the circuit 35 for the transcutaneous electro nerve stimulator 30 are possible, and mainly concern the control of the wave form shapes, frequencies, voltages and current values of the output electrodes. Since the transcutaneous electro nerve stimulator 30 is a well-known technology, it requires no further description; additional information about the transcutaneous electro nerve stimulator 30 may be found in U.S. Pat. No. 4,598,713; No. 4,210,151 and No. 3,817,254.

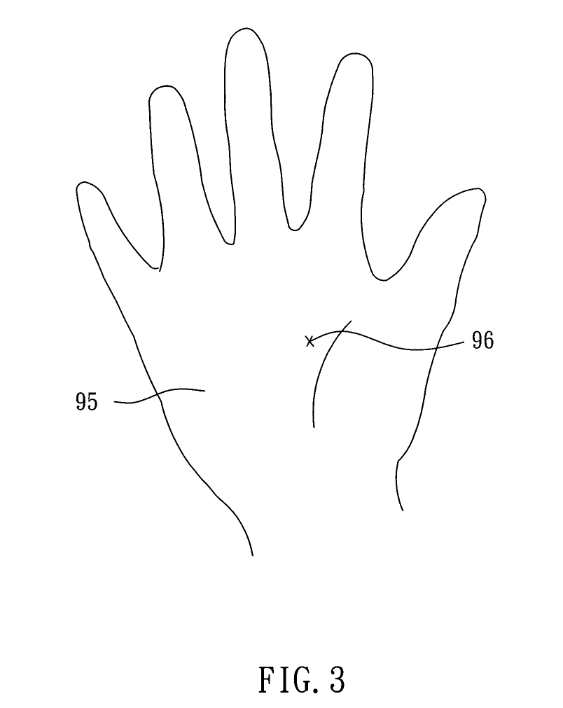

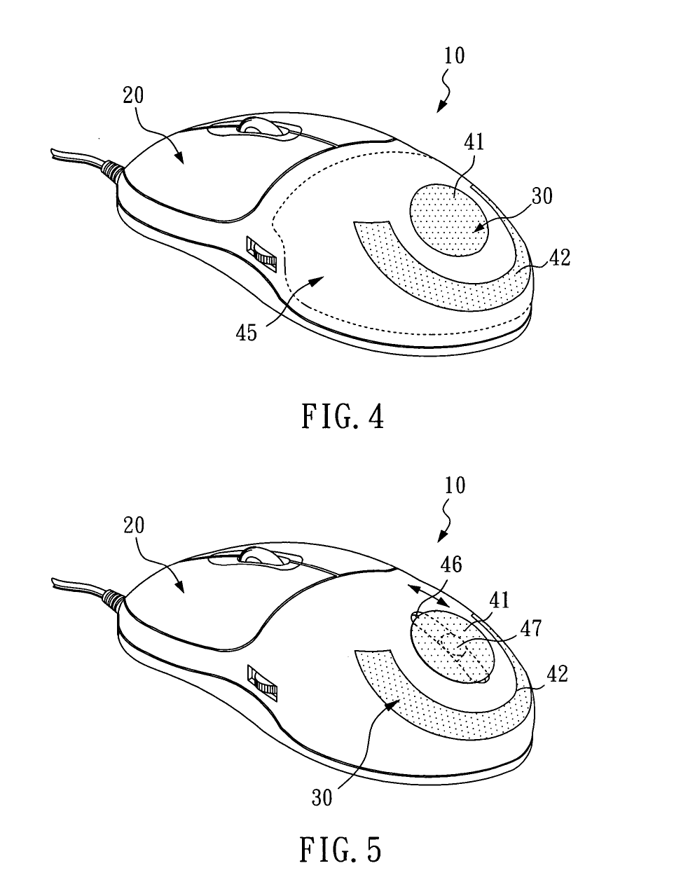

The computer mouse device 20 and the transcutaneous electro nerve stimulator 30 are both placed in a common housing 40, and this common housing may be composed of several housings. The common housing 40 comprises a hand-holding region 45 (referenced as an area within a dashed-line shown in FIG. 3 and FIG. 4). The hand-holding region 45 can be held by a palm 95 of a user, and the hand-holding region 45 is located at a rear end of the present invention computer mouse 10. In this embodiment, a first conductive region 41 and a second conductive region 42 are placed on surface of the common housing 40. The first conductive region 41 and the second conductive region 42 can be formed from a conductive material (such as a metallic material) coated on the common housing 40, and the first output electrode 31 and the second output electrode 32 of the transcutaneous nerve electro stimulator 30 are respectively connected to the first conductive region 41 and the second conductive region 42.

The first conductive region 41 and the second conductive region 42 should be located in the hand-holding region 45.

The first conductive region 41 and the first output electrode 31 should not only be simply in the hand-holding region 45, but should also be aligned with a position of the lao-gong point 96 in the palm 95 of the user, so that when the user is holding the device 10, the the lao-gong point 96 of the palm 95 can touch the first conductive region 41/the first output electrode 31, as shown in FIG. 3 (the lao-gong point are one of points on human body according the tradition Chinese medical science which are also the points for acupuncture).

Moreover, the first conductive region 41 can be raised slightly to ensure the lao-gong point 96 of the palm 95 can touch the first conductive region 41. The first conductive region 41/the first output electrode 31 can also be a flexible protrusion (not shown) so as to be more comfortable for the user.

The second conductive region 42 is also touched by the palm 95 of the user, and in this embodiment, the second conductive region 42 is annular and located in the hand-holding region 45; however, the second conductive region 42 may also be located in regions that the user's thumb or little finger may touch, rather than in the hand-holding region 45.

A switch 39 (such as a slide switch or a roller switch) for controlling the transcutaneous electro nerve stimulator 30 is electrically connected to the circuit 35, and the switch 39 may also adjust the amount of current. Of course, a software program can be installed in the computer 90 that may enable the user to control the transcutaneous electro nerve stimulator 30 by operating the software program, and in such an embodiment there would be no need for the switch 39.

FIG. 4 is a perspective drawing of a second embodiment of the computer mouse with transcutaneous nerve electro stimulation capabilities according to the present invention. In the second embodiment, the second conductive region 42 has a different shape.

FIG. 5 is a perspective drawing of a third embodiment of a computer mouse with transcutaneous electro nerve stimulation capabilities according to the present invention. In the third embodiment, the first conductive region 41 can be located at a different position to accommodate different palm sizes so as to ensure the lao-gong point 96 in the palm of the user can touch the first conductive region 41. A sliding block 47 can be installed under the first conductive region 41 to permit movement of the first conductive region 41, and a corresponding track 46 can be installed on the common housing 40 so that the sliding block 47 may move along the track 46.

Although the present invention has been explained in relation to its preferred embodiment, it is to be understood that many other possible modifications and variations can be made without departing from the spirit and scope of the invention as hereinafter claimed. For example, the output characteristics of the transcutaneous electro nerve stimulator, such as wave form shape, frequency, voltage and current amounts are all changeable; control buttons, or even a little monitor to provide an operational interface, may be provided to control these characteristics. Furthermore, a software program can be installed on the computer (or accessed via a network) that may enable the user to select different output characteristics; this is particularly true when the computer mouse has an input/output interface (such as a USB connector), since the computer can then send control signals to the transcutaneous electro nerve stimulator.

What is claimed is:

1. A computer mouse with transcutaneous electro nerve stimulation capabilities adapted for electrical connection to a computer, the device comprising:
a computer mouse device;
a transcutaneous electro nerve stimulator (T.E.N.S.) comprising a first output electrode and a second output electrode; and
a common housing for containing the computer mouse device and the transcutaneous electro nerve stimulator.

2. The computer mouse with transcutaneous electro nerve stimulation capabilities as claimed in claim 1, wherein the common housing comprises a hand-holding region, and the first output electrode is located in the hand-holding region.

3. The computer mouse with transcutaneous electro nerve stimulation capabilities as claimed in claim 1, wherein the common housing further comprises a first conductive region and a second conductive region, and the first output electrode and the second output electrode are respectively connected to the first conductive region and the second conductive region.

4. The computer mouse with transcutaneous electro nerve stimulation capabilities as claimed in claim 3, wherein the common housing comprises a hand-holding region, and the first conductive region is located in the hand-holding region.

5. The computer mouse with transcutaneous electro nerve stimulation capabilities as claimed in claim 4, wherein the second conductive region is located in the hand-holding region.

6. The computer mouse with transcutaneous electro nerve stimulation capabilities as claimed in claim 1, wherein the first output electrode can be changed to a different location.

7. The computer mouse with transcutaneous electro nerve stimulation capabilities as claimed in claim 3, wherein the first conductive region can be changed to a different location.
